(19) United States
(12) Patent Application Publication
(10) Pub. No.: US 2023/0001033 A1
(43) Pub. Date: Jan. 5, 2023

(54) NITROGEN GENERATION AND SANITIZING SYSTEM

(71) Applicant: BIOMIST, INC., Wheeling, IL (US)

(72) Inventors: Jay L. SHULER, Prairie View, IL (US); Robert L. COOK, II, Mt. Prospect, IL (US)

(73) Assignee: BIOMIST, INC., Wheeling, IL (US)

(21) Appl. No.: 17/847,956

(22) Filed: Jun. 23, 2022

Related U.S. Application Data
(60) Provisional application No. 63/214,055, filed on Jun. 23, 2021.

(51) Int. Cl. A61L 2/22; B01D 53/22; B01D 46/00; B01D 53/26

(52) U.S. Cl. CPC A61L 2/22; B01D 53/22; B01D 46/003; B01D 53/26; A61L 2101/34

(57) ABSTRACT

A sanitizing spray system includes a Nitrogen generation system that produces Nitrogen used as a carrier gas source for spraying an alcohol-based disinfecting and sanitizing composition without flashing.

### CROSS-REFERENCE TO RELATED APPLICATIONS

This application claims the benefit of U.S. Provisional Patent Application No. 63/214,055, filed Jun. 23, 2021, which is incorporated by reference herein in its entirety.

### BACKGROUND OF THE DISCLOSURE

### 1. Field of the Disclosure

The present disclosure provides a Nitrogen generation and sanitizing system. More particularly, the present disclosure provides a non-flammable alcohol atomizing sanitizing and disinfecting system operable by generated Nitrogen as a carrier gas.

### 2. Description of Related Art

Sanitizing and disinfecting systems that spray alcohol from a sprayer use carbon dioxide or Nitrogen as a carrier gas because these gases do not react with the alcohol, thus avoiding the risk of ignition while or immediately after spraying. The carbon dioxide gas or Nitrogen gas is provided in compressed liquified form from a gas cylinder.

These systems are generally limited to one sprayer because of carrier gas flow restrictions due to the temperature and volume changes of the gases upon exiting the cylinder. Further, frosting on the sprayer can occur and seizing after prolonged use.

Compressed gas cylinder replacement requires downtime. The cylinders require storage space and maintenance. Importantly, the cylinder must constantly be refilled. Disruptions with deliveries can interrupt operations. Not only, but special care must also be taken to handle compressed gases.

Accordingly, it has been determined by the present disclosure that there is a continuing need for a sanitizing and disinfecting system that overcomes, alleviates, and/or mitigates one or more of the aforementioned and other deleterious effects of prior devices.

### SUMMARY OF THE DISCLOSURE

The present disclosure provides a system and method for spraying a non-flammable alcohol atomized spray.

The present disclosure provides a system and method that does not require liquified compressed gas.

The present disclosure provides a sanitizing spray system that includes a Nitrogen generation system that produces Nitrogen used as a carrier gas source for spraying an alcohol-based disinfecting composition without flashing.

A sanitizing system includes a container for an alcohol-based disinfecting composition, a sprayer, and a Nitrogen generator producing 99.5% pure Nitrogen as a carrier gas for the sprayer to dispense the alcohol-based disinfecting composition. The container, sprayer, and Nitrogen generator are in fluid communication and configured to dispense the alcohol-based disinfecting composition and Nitrogen carrier gas.

In examples according to the present disclosure, the Nitrogen generator includes a dryer, an air compressor, a compressed air tank, a membrane filter, and a compressed Nitrogen storage tank. The Nitrogen generator can also include a controller to control the sanitizing system.

In examples according to the present disclosure, the Nitrogen generator can be configured to generate Nitrogen for storage in a Nitrogen storage tank at a pressure of at least 10 psi.

In examples according to the present disclosure, the generated Nitrogen and alcohol-based disinfecting composition are sprayed or dispensed in a ratio of Nitrogen to alcohol-based disinfecting composition that is at least 750 to 1.

In examples according to the present disclosure, the sanitizing system has at least one 0.01 microns coalescing filter to filter ambient air. The ambient air and/or generated Nitrogen can be purified to least 99.9% pure Nitrogen.

In examples according to the present disclosure, the alcohol-based disinfecting composition comprises at least 60% of an alcohol. The alcohol-based disinfecting composition can be at least 70% ethyl or isopropyl alcohol.

In certain examples, the sanitizing system according to the present disclosure can be mounted on the cart.

In other examples, the system can be disposed in a housing and the housing can have one or more straps or handles.

The present disclosure also provides a method for sanitizing surfaces with an alcohol-based disinfecting composition.

The method includes generating Nitrogen from ambient air with a nitrogen generator and pressurizing a container having a spray nozzle and containing the alcohol-based disinfecting composition with the generated Nitrogen to cause the Nitrogen and alcohol-based disinfecting composition to be dispensed from the nozzle.

The method can further include, compressing the ambient air with a compressor, filtering the compressed air with a membrane filter to yield at least 99.5% Nitrogen, storing the compressed air in a storage tank prior to the filtering, storing the Nitrogen in a storage tank prior to pressurizing the container, drying the ambient air to at least about 5% relative humidity, maintaining at least a 750:1 Nitrogen to alcohol-based disinfecting composition ratio, and/or filtering the ambient air to remove particles greater than 0.01 micron.

### BRIEF DESCRIPTION OF THE DRAWINGS

FIG. 1 shows a spray system according to the present disclosure.

FIG. 2 is a left side view of a Nitrogen system for the spray system shown in FIG. 1.

FIG. 3 is a front side view of a Nitrogen system for the spray system shown in FIG. 1.

FIG. 4 is a right side view of a Nitrogen system for the spray system shown in FIG. 1.

FIG. 5 is a rear side view of a Nitrogen system for the spray system shown in FIG. 1.

FIG. 6 shows a cart having the spray system shown in FIG. 1.

FIG. 7 shows a backpack having the spray system shown in FIG. 1.

### DETAILED DESCRIPTION OF THE DISCLOSURE

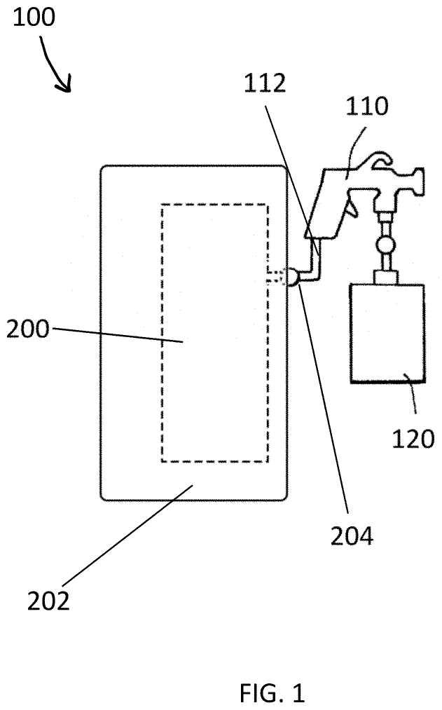

Referring to the drawings and in particular to FIG. 1, there is shown a spray system for spraying an alcohol-based disinfecting composition according to the present disclosure and generally referenced by the numeral 100, hereinafter spray system 100.

Spray system 100 includes a Nitrogen generation system, Nitrogen system 200, that produces or generates Nitrogen used as a carrier gas source for spraying the alcohol-based disinfecting composition. The system can be used to sanitize surfaces by dispensing the alcohol-based disinfecting composition from a sprayer and onto the surfaces, for example.

As used herein, the term Nitrogen generation refers to air-to-nitrogen production complexes.

As used herein, the term “sprayer” refers to a device or apparatus for atomizing a liquid and delivering the liquid to a work surface. Such a device or apparatus is commonly referred to as a “spray gun”. Sprayers vary in their configuration, size, weight and internal composition. Various nozzles and nozzle configurations result in different spray patterns. Non-limiting examples include siphon-fed spray guns, pressure-fed spray guns, and gravity-fed spray guns.

Nitrogen system 200 can be disposed in a housing 202.

Spray system 100 also includes a sprayer 110 in fluid communication with a container 120 for the alcohol-based disinfecting composition. A hose 112 provides fluid communication between container 120 and an outlet 204 of Nitrogen system 200.

In operation, upon actuation of sprayer 110, Nitrogen output by Nitrogen system 200 flows through hose 112 into container 120. By function of negative pressure, the alcohol-based disinfecting composition is expelled from sprayer 110 together with Nitrogen gas generated by Nitrogen system 200 as a carrier gas.

An alcohol-based disinfecting composition can contain 50% to 99%, preferably 60% to 80%, and most preferably 65 to 80% of alcohol. For example, the alcohol-based disinfecting composition can contain 70% ethyl or isopropyl alcohol.

The composition can be a hospital-grade alcohol-based food contact and non-food contact surface sanitizer.

The alcohol-based disinfecting composition can include ethyl or isopropyl alcohol. The alcohol-based disinfecting composition can include ethyl or isopropyl alcohol and a quaternary ammonium or other sanitizing chemicals or compounds.

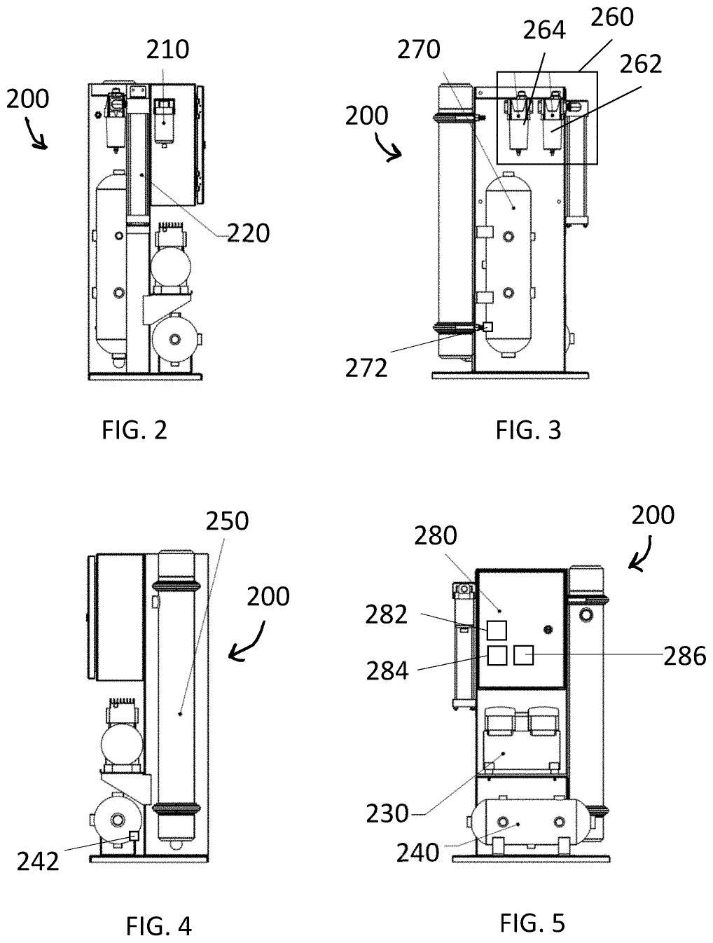

Referring now to FIGS. 2 to 5, Nitrogen system 200 includes, in operable fluid connection, a pre-filter 210, an air dryer 220, an air compressor 230, a compressed air tank 240, a membrane filter 250, a filter 260, a compressed Nitrogen storage tank 270, and a controller 280.

Nitrogen system 200 generates purified Nitrogen (99.5%) operable as a carrier gas in a spray system such as spray system 100.

In operation, air compressor 230 draws ambient air through air dryer 220 through a one-way valve (not shown) and into compressed air tank 240. The one-way valve prevents backflow leakage when air compressor 230 is not pumping.

Pressure switches that are part of or in operative communication with controller 280 are configured to monitor a pressure of compressed air tank 240 and compressed Nitrogen storage tank 270. These switches detect high and low pressure.

A high-pressure switch 282 closes when a pressure in compressed air tank 240 reaches 90 psig. Controller 280 causes air compressor 230 to turn off or deenergize when high-pressure switch 282 closes. A low-pressure switch 284 closes when a pressure in compressed air tank 240 drops below from about 30 psig to 50 psig, and preferably below from about 30 to 50 psig. Advantageously, these pressures have been found top provide efficient use of the compressor. Controller 280 causes air compressor 230 to energize or turn on when low-pressure switch 284 closes.

Controller 280 is configured to open a solenoid valve 242 when low-pressure switch 284 is closed for a predetermined time, for example 10, 15, 20, or 30 or more seconds. Controller 280 is also configured to open solenoid valve 242 when low-pressure switch 284 is open.

When solenoid valve 242 opens, compressed air stored in compressed air tank 240 passes through pre-filter 210 that filter out particles greater than about 100 microns. Thereafter, the compressed air passes through air dryer 220.

Air dryer 220 removes moisture. Water separator can be, for example, a desiccant dryer. Air dryer 220 removes water to at most about 5% relative humidity, preferably at most about 3%, more preferably at most about 1%, and most preferably, at most about 0.5%.

Compressed air tank 240 is a pressure vessel for storing the ambient air at a higher pressure than ambient. For example, compressed air tank can store air at, for example, 50 psi to 90 psi, 40 to 100 psi, 30 to 120 psi, or combinations of these ranges.

Air from compressed air tank 240 flows though filter 260. Air then flows through membrane filter 250 and into compressed Nitrogen storage tank 270.

Filter 260 includes an at least 0.01 micron filter, pre-filter 210. In embodiments, filter 260 can also include one or more filters 264 upstream of pre-filter 210. Filter 264 filters larger particles than filter 262. Filter 262 can be, for examples, a 0.03 micron filter.

Membrane filter 250 filters the air to be operable as a carrier gas for spray system 100, namely to at least about 99.5% pure Nitrogen. This purity is important to prevent combustion. For example, membrane filter 250 filters the air to be at least about 99.5% pure Nitrogen, more preferably at least about 99.9% pure Nitrogen, and most preferably at least about 99.99% pure Nitrogen.

Membrane filter can be a hollow fiber membrane that provides a minimum flow of 80 LPM @ 40 psi or a constant flow rate and pressure to the spray nozzle/nozzles that maintains the at least 750:1 gas to solution spray.

In embodiments, membrane filter 250 can be substituted with a pressure swing adsorption filter. Pressure swing adsorption filters use carbon molecular sieves to adsorb oxygen molecules. Advantageously, a pressure swing adsorption filter can filter air to up to at least about 99.99% Nitrogen.

Compressed Nitrogen storage tank 270 is a pressure vessel for storing Nitrogen output by membrane filter 250 prior to and during use of spray system 100.

A one-way valve (not shown) between membrane filter 250 and compressed Nitrogen storage tank 270 prevents reverse flow through the membrane filter which could damage the membrane.

As discussed above, controller 280 is configured to monitor a pressure of compressed Nitrogen storage tank 270. When a pressure of compressed Nitrogen storage tank 270 reaches at least 25, 30, 40 or more psi Nitrogen storage pressure switch 286 closes. Controller 280 opens a solenoid valve 272 for enabling a flow of Nitrogen when the Nitrogen storage pressure switch 286 has been closed for a minimum of 10, 15, 20, or 30 or more seconds.

Solenoid valve 272 is in fluid communication with compressed Nitrogen storage tank 270 and outlet 204.

Nitrogen the flows into hose 112 into container 120. As discussed above, by function of negative pressure, the alcohol-based disinfecting composition is expelled from sprayer 110 together with Nitrogen gas generated by Nitrogen system 200 as a carrier gas.

Controller 280 is comprised of logic and circuitry configured to operate spray system 100.

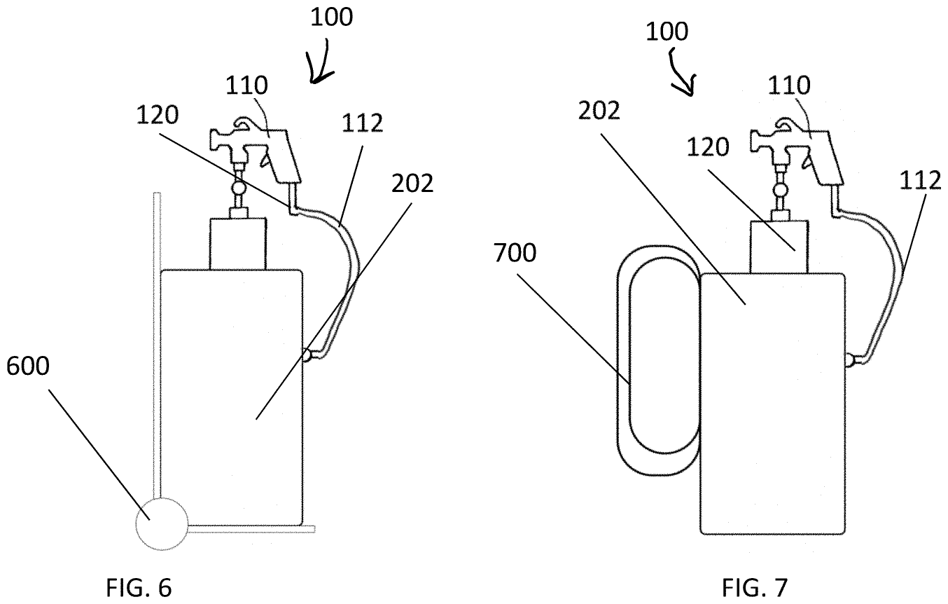

Referring to FIG. 6, spray system 100 is incorporated in a cart 600. Cart 600 enables the system to be rolled around during use.

Referring now to FIG. 7, housing 202 can include one or more handles or straps, members 700. Members 700 enable housing 202 to be carried like bag or backpack.

### Experimental

A flame flash test was performed with various nozzles. A torch flame about 3″ from the spray nozzle is moved into the spray stream. To pass the test, the spray stream should extinguish the flame. If instead the flame flashes, the test fails. The results are summarized in Table 1. Comparative data with Carbon Dioxide is shown in Table 2. The present disclosure has found that a Nitrogen generation system and sprayer has performance comparable to prior Carbon Dioxide tank based systems.

The first column lists the various nozzles tested. The second column shows the Nitrogen pressure from the Nitrogen generation system according to the present disclosure. The third and fifth columns show Nitrogen flow in liters per minute and cubic feet per minute, respectively. The fourth and sixth columns show disinfecting composition flow in liters per minute and cubic feet per minute, respectively. The seventh and final column is a ratio of parts carbon dioxide and Nitrogen to 1 part solution of disinfecting composition. Rows shown in bold face with underlines indicate continuous Nitrogen generation while running.

The present disclosure has found that a Nitrogen pressure under 30 psi impacts the Humphrey valve solution flow.

TABLE 1

| | N₂- | N₂ | Solution | N₂ | Solution | Ratio N₂ to |
|---|---|---|---|---|---|---|
| | PSI | LPM | ml PM | CFM | CFM | Solution |
| SS-20 Spray Gun | 10 | 37 | 0 | 1.31 | 0.0000 | |
| DeVilbiss | 20 | 63 | 0 | 2.23 | 0.0000 | |
| For Manual | 30 | 91 | 72 | 3.21 | 0.0025 | 1264 |
| Spraying | 40 | 124 | 123 | 4.38 | 0.0043 | 1008 |
| Volume Max | 50 | 146 | 138 | 5.16 | 0.0049 | 1058 |
| Spray Wide | | | | | | |
| SS-10/SS-5 | 10 | 25 | 14 | 0.88 | 0.0005 | 1786 |
| Binks 190 | 20 | 44 | 44 | 1.55 | 0.0016 | 1000 |
| SS-40100/ | 30 | 55 | 60 | 1.94 | 0.0021 | 917 |
| 120-ENP | 40 | 70 | 88 | 2.47 | 0.0031 | 795 |
| For Manual | 50 | 93 | 104 | 3.29 | 0.0037 | 894 |
| Spraying | | | | | | |

| | N₂- | N₂ | Solution | | Solution | Ratio N₂ to | |
|---|---|---|---|---|---|---|---|
| | PSI | LPM | Floz | N₂CFM | CFM | Solution | |
| BETE - EF150 | 10 | 25 | 0 | 0.88 | 0.0000 | | |
| FC4 - AC1001 | 20 | 41 | 0.8 | 1.45 | 0.0008 | 1733 | |
| Fluid Pressure 10 | 30 | 56 | 1.2 | 1.98 | 0.0013 | 1578 | 2x |
| For Machine | 40 | 63 | 1.7 | 2.23 | 0.0018 | 1253 | |
| Spraying | 50 | 82 | 1.3 | 2.90 | 0.0014 | 2133 | |
| Wide Flat Spray | | | | | | | |
| BETE - EF150 | 20 | 42 | 2.5 | 1.48 | 0.0026 | 568 | |
| FC4 - AC1001 | 30 | 56 | 3.6 | 1.98 | 0.0038 | 526 | |
| Fluid Pressure 15 | 40 | 70 | 3.6 | 2.47 | 0.0038 | 658 | |
| | 50 | 82 | 3.6 | 2.90 | 0.0038 | 770 | |
| BETE - EF150 | 20 | 42 | 1.6 | 1.48 | 0.0017 | 888 | |
| FC4 - AC1001 | 30 | 56 | 2.6 | 1.98 | 0.0027 | 728 | ** Flame |
| Fluid Pressure 20 | 40 | 70 | 4.7 | 2.47 | 0.0049 | 504 | ** Flame |
| | 50 | 82 | 4.6 | 2.90 | 0.0048 | 603 | ** Flame |
| Pipe Spraying | 20 | 38 | 2.5 | 1.34 | 0.0026 | 514 | ** Flame |
| BETE - AD150 | 30 | 51 | 1 | 1.80 | 0.0010 | 1725 | |
| FC2 - AC1602 | 40 | 63 | 0.6 | 2.23 | 0.0006 | 3551 | |
| Fluid Pressure 10 | 50 | 85 | 0 | 3.00 | 0.0000 | | |
| BETE - AD150 | 20 | 41 | 1.3 | 1.45 | 0.0014 | 1067 | |
| FC2 - AC1602 | 30 | 54 | 1.7 | 1.91 | 0.0018 | 1074 | |
| Fluid Pressure 15 | 40 | 63 | 1.8 | 2.23 | 0.0019 | 1184 | |
| | 50 | 85 | 0.7 | 3.00 | 0.0007 | 4107 | |
| BETE - AD150 | 20 | 38 | 2.4 | 1.34 | 0.0025 | 536 | ** Flame |
| FC2 - AC1602 | 30 | 48 | 5.2 | 1.70 | 0.0054 | 312 | ** Flame |
| Fluid Pressure 20 | 40 | 59 | 3.8 | 2.08 | 0.0040 | 525 | ** Flame |
| | 50 | 85 | 2.8 | 3.00 | 0.0029 | 1027 | |

TABLE 2

| | Formula | | | | | |
|---|---|---|---|---|---|---|
| | D2 | CO₂ | Solution | CO₂- | Solution | Ratio |
| 50 ft Hose | CO₂-PSI | LPM | mIM | CFM | CFM | N to S |
| SS-20 Spray Gun | 10 | 40 | 0 | 1.41 | 0.0000 | |
| DeVilbiss | 20 | 68 | 0 | 2.40 | 0.0000 | |
| For Manual | 30 | 90 | 78 | 3.18 | 0.0028 | 1154 |
| Spraying | 40 | 106 | 134 | 3.74 | 0.0047 | 791 |
| Volume Max | 50 | 135 | 160 | 4.77 | 0.0057 | 844 |
| Spray Wide | | | | | | |
| SS-10/SS-5 | 10 | 27 | 14 | 0.95 | 0.0005 | 1929 |
| Binks 190 | 20 | 39 | 34 | 1.38 | 0.0012 | 1147 |
| SS-40100/ | 30 | 51 | 74 | 1.80 | 0.0026 | 689 |
| 120-ENP | 40 | 72 | 96 | 2.54 | 0.0034 | 750 |
| For Manual | 50 | 83 | 106 | 2.93 | 0.0037 | 783 |
| Spraying | | | | | | |

| | Formula | | | | | | |
|---|---|---|---|---|---|---|---|
| | D2 | CO₂ | Solution | CO₂- | Solution | Ratio | |
| 10 ft Hose | CO₂-PSI | LPM | mIM | CFM | CFM | N to S | |
| SS-20 Spray Gun | 10 | 50 | 0 | 1.77 | 0.0000 | | Spurts/ |
| DeVilbiss | | | | | | | No Flow |
| For Manual | 20 | 78 | 60 | 2.76 | 0.0021 | 1300 | |
| Spraying | 30 | 102 | 126 | 3.60 | 0.0044 | 810 | |
| Volume Max | 40 | 121 | 162 | 4.27 | 0.0057 | 747 | |
| Spray Wide | 50 | 142 | 205 | 5.02 | 0.0072 | 693 | |
| SS-10/SS-5 | 10 | 27 | 24 | 0.95 | 0.0008 | 1125 | |
| Binks 190 | 20 | 43 | 56 | 1.52 | 0.0020 | 768 | |
| SS-40100/ | 30 | 57 | 80 | 2.01 | 0.0028 | 713 | |
| 120-ENP | 40 | 70 | 98 | 2.47 | 0.0035 | 714 | |
| For Manual | 50 | 83 | 108 | 2.93 | 0.0038 | 769 | |
| Spraying | | | | | | | |

Table 3 shows test data using an alcohol solution of 99%.

TABLE 3

| | N2- | Nitrogen | Solution | Nitrogen | Solution | Ratio |
|---|---|---|---|---|---|---|
| | PSI | LPM | mIM | CFM | CFM | N to S |
| SS-20 | 10 | 54 | 0 | 1.91 | 0.0000 | |
| Spray Gun | 20 | 82 | 56 | 2.90 | 0.0020 | 1464 |
| DeVilbiss | 30 | 115 | 132 | 4.06 | 0.0047 | 871 |
| For Manual | 40 | 130 | 156 | 4.59 | 0.0055 | 833 |
| Spraying | 50 | 152 | 190 | 5.37 | 0.0067 | 800 |
| Volume Max | | | | | | |
| Spray Wide | | | | | | |
| SS-10/SS-5 | 10 | 32 | 32 | 1.13 | 0.0011 | 1000 |
| Binks 190 | 20 | 48 | 58 | 1.70 | 0.0020 | 828 |
| SS-40100/ | 30 | 58 | 88 | 2.05 | 0.0031 | 659 |
| 120-ENP | 40 | 75 | 102 | 2.65 | 0.0036 | 735 |
| For Manual | 50 | 90 | 116 | 3.18 | 0.0041 | 776 |
| Spraying | | | | | | |

The present disclosure has found that Nitrogen can be generated at ambient temperature and used as a carrier gas for spraying alcohol-based disinfecting composition at a ratio of Nitrogen to Solution over 750 to avoid flame flashing.

Advantageously, providing spray system 100 Nitrogen at ambient temperature eliminates frosting.

As used herein, the term “substantially” means the complete or nearly complete extent or degree of an action, characteristic, property, state, structure, item, or result. For example, an object that is “substantially” enclosed means that the object is either completely enclosed or nearly completely enclosed. The exact allowable degree of deviation from absolute completeness can in some cases depend on the specific context. However, generally, the nearness of completion will be to have the same overall result as if absolute and total completion were obtained.

As used herein, the term “comprising” means “including, but not limited to; the term “consisting essentially of” means that the method, structure, or composition includes steps or components specifically recited and may also include those that do not materially affect the basic novel features or characteristics of the method, structure, or composition; and the term “consisting of” means that the method, structure, or composition includes only those steps or components specifically recited.

As used herein, the term “about” is used to provide flexibility to a numerical range endpoint by providing that a given value can be “a little above” or “a little below” the endpoint. Further, where a numerical range is provided, the range is intended to include all numbers within the numerical range, including the end points of the range.

While the present disclosure has been described with reference to one or more exemplary embodiments, it will be understood by those skilled in the art, that various changes can be made, and equivalents can be substituted for elements thereof without departing from the scope of the present disclosure. In addition, many modifications can be made to adapt a particular situation or material to the teachings of the present disclosure without departing from the scope thereof. Therefore, it is intended that the present disclosure will not be limited to the particular embodiments disclosed herein, but that the disclosure will include all aspects falling within the scope of a fair reading of appended claims.

What is claimed is:

1. A sanitizing system comprising:
a container for an alcohol-based disinfecting composition;
a sprayer; and
a Nitrogen generator producing 99.5% pure Nitrogen as a carrier gas for the sprayer to dispense the alcohol-based disinfecting composition,
wherein the container, sprayer, and Nitrogen generator are in fluid communication and configured to dispense the alcohol-based disinfecting composition and Nitrogen carrier gas.

2. The sanitizing system of claim 1, wherein the Nitrogen generator comprises: a dryer, an air compressor, a compressed air tank, a membrane filter, and a compressed Nitrogen storage tank.

3. The sanitizing system of claim 2, wherein the Nitrogen generator further comprises a controller.

4. The sanitizing system of claim 2, wherein the Nitrogen generator generates Nitrogen for storage in the compressed Nitrogen storage tank at a pressure of at least 10 psi.

5. The sanitizing system of claim 2, wherein the generated Nitrogen and alcohol-based disinfecting composition are in a ratio of Nitrogen to alcohol-based disinfecting composition that is at least 750 to 1.

6. The sanitizing system of claim 2, further comprising at least one 0.01 microns coalescing filter.

7. The sanitizing system of claim 1, wherein the Nitrogen is at least 99.9% pure Nitrogen.

8. The sanitizing system of claim 1, wherein the alcohol-based disinfecting composition comprises at least 60% of an alcohol.

9. The sanitizing system of claim 1, wherein the alcohol-based disinfecting composition comprises at least 70% ethyl or isopropyl alcohol.

10. The sanitizing system of claim 1, further comprising: a cart, wherein the sanitizing system is mounted on the cart.

11. The sanitizing system of claim 1, wherein Nitrogen generator is disposed in a housing and the housing comprises one or more straps or handles.

12. A method for sanitizing surfaces with an alcohol-based disinfecting composition, the method comprising:
generating Nitrogen from ambient air with a nitrogen generator; and
pressurizing a container of the alcohol-based disinfecting composition having a spray nozzle with the generated Nitrogen to cause the Nitrogen and alcohol-based disinfecting composition to be dispensed from the spray nozzle.

13. The method of claim 12, further comprising:
compressing the ambient air with a compressor; and
filtering the compressed air with a membrane filter to yield at least 99.5% Nitrogen.

14. The method of claim 13 further comprising:
storing the compressed air in a storage tank prior to the filtering.

15. The method of claim 12, further comprising:
storing the Nitrogen in a storage tank prior to pressurizing the container.

16. The method of claim 12, further comprising:
drying the ambient air to at least about 5% relative humidity.

17. The method of claim 12, further comprising:
maintaining at least a 750:1 Nitrogen to alcohol-based disinfecting composition ratio.

18. The method of claim 12, further comprising:
filtering the ambient air to remove particles greater than 0.01 micron.
